(19) United States
(12) Patent Application Publication
(10) Pub. No.: US 2023/0126453 A1
(43) Pub. Date: Apr. 27, 2023

(54) IMAGING OF BRAIN LESIONS

(71) Applicant: Blue Earth Diagnostics Limited, Oxford (GB)

(72) Inventor: Eugene TEOH, Oxford (GB)

(73) Assignee: Blue Earth Diagnostics Limited, Oxford (GB)

(21) Appl. No.: 17/508,007

(22) Filed: Oct. 22, 2021

(51) Int. Cl. A61B 6/00; A61K 51/04; A61B 6/03

(52) U.S. Cl. CPC A61B 6/481; A61B 6/501; A61K 51/0406; A61B 6/037

(57) ABSTRACT

The present disclosure relates to methods of administering [¹⁸F]-FACBC. The present disclosure also relates to use of [¹⁸F]-FACBC in methods for imaging, diagnosing, and monitoring of brain lesions.

### TECHNICAL FIELD

The present disclosure relates to methods of administering [₁₈F]-FACBC. The present disclosure also relates to use of [¹⁸F]-FACBC in methods for imaging and diagnosing brain lesions.

### BACKGROUND

Brain metastases are the most common intracranial tumor in adults, occurring in up to 40% of patients with cancer, with approximately 200,000 patients affected each year in the USA. Following localized treatment of brain metastases (increasingly, stereotactic radiosurgery [SRS] alongside neurosurgical resection), close follow-up with serial magnetic resonance imaging (MRI) of the brain is performed to evaluate for recurrent disease. Conventional MRI is currently recommended as the main imaging test (NCCN, 2019) following localized treatment, as it is widely available and offers high spatial resolution, with presence of recurrent disease suggested by increased contrast enhancement (CE) depicting anatomical/structural information. However, conventional MRI (CE-T1 and fluid attenuated inversion recovery [FLAIR]/T2-weighted sequences) has limited specificity due to the incidence of treatment-related changes, including radiation necrosis. These treatment-related changes have similar appearance to true recurrence of disease on conventional MRI, including CE, origin near the primary tumor site, vasogenic edema, growth over time, and mass effect.

No specific feature or combination of features on conventional MRI has been established to differentiate between disease recurrence and treatment-related changes including radiation necrosis and pseudoprogression. Specificity of conventional MRI to diagnose recurrent tumor after SRS using visual reads has been reported to be as low as 19%, with attempts at validating permutations of neuroradiologist-defined measurements reporting specificities of 32% to 41%. Alongside the estimated 25% incidence rate of radiation necrosis, rates of true local recurrence of disease are similar, ranging from 27% to 31%. Therefore, the true prevalence of recurrent disease post-radiotherapy where conventional MRI indicates the possibility of recurrence, can be estimated to be approximately 50%.

Given this area of great diagnostic unmet need, accurate imaging to differentiate disease recurrence from treatment-related changes is valuable for several reasons:
Identifying treatment-related changes is important to avoid unneeded treatment (e.g., surgery) and erroneously premature termination of potentially effective treatment.
Accurate scans can inform the management decision of cessation of non-effective treatments, to minimize morbidity from treatment side effects and reduce the economic burden.
Timely diagnosis of true recurrence can facilitate prompt stratification of patients to further therapies, which may maximize therapeutic benefit and clinical outcome.
Given the high morbidity and mortality of patients with brain metastases and, therefore, the need for high quality clinical research, such imaging may be pivotal in determining suitability for clinical trial entry, and accurate characterization of investigational therapeutic efficacy. This particular need for certainty on optimal clinical trial endpoints has been recognized by the Food and Drug Administration (FDA) and National Brain Tumor Society (NBTS), leading to recent joint efforts to stimulate much needed research and development in this area (FDA/NBTS, 2019).
Aiding the physician to risk-stratify continuation of a therapeutic regimen (where treatment-related changes can be confidently diagnosed). This may be of particular value in the context of a treatment with a significant side effect profile.

[¹⁸F]-fluoro-2-deoxy-glucose fluoro-2-deoxy-glucose (FDG) is a successful PET imaging agent for the detection and localisation of many forms of cancer. However, FDG-PET has been found to have less sensitivity and/or specificity for assessment of some types of cancer, for example, brain tumors.

Metastasis involves a complex series of steps in which cancer cells leave the primary tumor site and migrate to other parts of the body via the bloodstream or the lymphatic system. The new occurrences of tumor thus generated are referred to as a metastatic lesion or metastasis. Metastatic lesions are very common in the late stages of cancer and are a major cause of death from solid lesions. There are known difficulties associated with diagnosing and monitoring metastatic cancer, for example, obtaining images at different time points with enough accuracy to enable comparison is challenging.

Thus there is a need for a method of imaging which allows for reproducible, reliable imaging for detection and monitoring of metastatic cancer in the brain.

### SUMMARY

Aspects of the present disclosure relate to methods of diagnosing brain lesions, e.g., analysing potential lesions to determine whether they are brain lesions, in a subject by administering [¹⁸F]-FACBC for improved PET imaging and more reliable diagnosis of cancer and the metastasis or recurrence thereof. For example, the present disclosure relates to methods of administering [¹⁸F]-FACBC for improved PET imaging and more reliable diagnosis of brain lesions and the metastasis or recurrence thereof. The present disclosure further relates to methods of diagnosing brain lesions and the metastasis or recurrence thereof, using the PET imaging agent [¹⁸F]-FACBC. Comparison of the uptake of [¹⁸F]-FACBC uptake in a potential lesion against [¹⁸F]-FACBC uptake in the blood pool and/or the parotid glands may allow for a more accurate diagnosis of the lesion than using MRI and/or CT alone.

In at least one aspect of the present disclosure, there is provided a method of using [¹⁸F]-FACBC (e.g., for diagnosing brain lesions)in a subject comprising the steps of:
(a) administering a detectable amount of [¹⁸F]-FACBC to the subject, e.g., administering an amount of [¹⁸F]-FACBC to the subject that is detectable by imaging;
(b) acquiring one or more positron emission tomography (PET) scan images of a head region of the subject, the head region including parotid glands and a blood pool;
(c) comparing [¹⁸F]-FACBC uptake in a potential lesion against [¹⁸F]-FACBC uptake in the blood pool and/or the parotid glands, e.g., analysing the one or more PET scan images by comparing [¹⁸F]-FACBC uptake in the potential lesion against [¹⁸F]-FACBC uptake in the blood pool of the head region, the parotid glands, or both the blood pool and the parotid glands; and
(d) determining whether the potential lesion is a brain lesion based on the analysis of step c).

Such exemplary aspects may include, for example, instructions for the subject to consume no food or calorie-containing drink during a period of time prior to the administration of the [¹⁸F]-FACBC, the subject having followed the instructions. The period of time may be, e.g., at least 4 hours or at least 6 hours prior to the administration of the [¹⁸F]-FACBC. Acquiring the PET scan image(s) may start 8 to 12 minutes after the administration of the [¹⁸F]-FACBC, e.g., 10 minutes after the administration of the [¹⁸F]-FACBC. In at least some cases, the [¹⁸F]-FACBC is administered as an intravenous bolus injection. An exemplary detectable amount of [¹⁸F]-FACBC is 185±20% MBq, according to some aspects herein.

Potential lesion [¹⁸F]-FACBC uptake (that is, uptake of [¹⁸F]-FACBC by the potential lesion) which is higher than blood pool and/or parotid glands [¹⁸F]-FACBC uptake may be indicative of the presence of brain lesions, e.g., indicative of the potential lesion being a brain lesion. For example, the [¹⁸F]-FACBC uptake in the potential lesion may be higher than the [¹⁸F]-FACBC uptake in the parotid glands, and step d) includes determining that the potential lesion is a brain lesion. Further, for example, the [¹⁸F]-FACBC uptake in the potential lesion may be higher than the [¹⁸F]-FACBC uptake in the blood pool, and step d) includes determining that the potential lesion is a brain lesion. In at least one example, the [¹⁸F]-FACBC uptake in the potential lesion is higher than both the [¹⁸F]-FACBC uptake in the blood pool and the [¹⁸F]-FACBC uptake in the parotid glands, and step d) includes determining that the potential lesion is a brain lesion. The brain lesion may be a cancer lesion, e.g., a metastatic cancer lesion. In some examples, the potential lesion may be suspicious for a recurrence of cancer in the subject. In at least one example, the [¹⁸F]-FACBC uptake in the potential lesion is lower than or equal to the [¹⁸F]-FACBC uptake in the blood pool or the [¹⁸F]-FACBC uptake in the parotid glands, and step d) includes determining that the potential lesion is not a brain lesion.

In some examples herein, the one or more PET scan images comprise a first PET scan image and at least one subsequent PET scan image, wherein analysing the one or more PET scan images in step c) comprises comparing intensities of [¹⁸F]-FACBC uptake in the first PET scan image and the at least one subsequent PET scan image to determine whether the intensity of [¹⁸F]-FACBC uptake in the at least one subsequent PET scan image has increased, remained constant, or decreased relative to the intensity of [¹⁸F]-FACBC uptake of the first PET scan image.

In some examples herein, the one or more PET scan images comprises a PET scan image at a first location and at least one PET scan image at a subsequent location, and wherein analysing the one or more PET scan images in step c) comprises comparing intensities of [¹⁸F]-FACBC uptake in the PET scan image at the first location and of [¹⁸F]-FACBC uptake in the at least one PET scan image at the subsequent location to determine whether the intensity of the [¹⁸F]-FACBC uptake in the at least one PET scan image at the subsequent location has increased, remained constant, or decreased relative to the intensity of the [¹⁸F]-FACBC uptake in the PET scan image at the first location.

In at least one aspect of the present disclosure, there is provided a method of using [¹⁸F]-FACBC, e.g., for diagnosing a brain lesion in a subject, the method comprising the steps of:
a) administering a detectable amount of [¹⁸F]-FACBC to the subject;
b) acquiring one or more positron emission tomography (PET) scan images of head region of the subject, the head region including parotid glands;
c) comparing [¹⁸F]-FACBC uptake in a potential lesion against [18F]-FACBC uptake in the blood pool and/or the parotid glands, e.g., analysing the one or more PET scan images by comparing [¹⁸F]-FACBC uptake in the potential lesion against [¹⁸F]-FACBC uptake in the blood pool of the head region, the parotid glands, or both the blood pool and the parotid glands;
wherein the subject is instructed to consume no food or calorie-containing drink during a period of time prior to the administration of [¹⁸F]-FACBC, e.g., wherein the subject has followed instructions to consume no food or calorie-containing drink during a period of time prior to the administration of the [¹⁸F]-FACBC; and
d) determining whether the potential lesion is a brain lesion based on the analysis of step c).

For example, [¹⁸F]-FACBC uptake in the potential lesion which is higher than the [18F]-FACBC uptake in the blood pool and/or the parotid glands may be indicative of the presence of the brain lesion, e.g., indicative of the potential lesion being a brain lesion.

In some embodiments of the present disclosure, the potential lesion is a brain lesion that contains diseased tissue and may be a cancer lesion, for example a metastatic cancer lesion.

Diagnosis of a brain lesion, for example a metastatic cancer lesion, may be performed where the potential lesion [¹⁸F]-FACBC uptake (that is, uptake of [¹⁸F]-FACBC by the potential lesion) is higher than parotid glands [¹⁸F]-FACBC uptake.

Diagnosis of a brain lesion, for example a metastatic cancer lesion may be performed where the potential lesion [¹⁸F]-FACBC uptake is higher than blood pool [¹⁸F]-FACBC uptake.

Diagnosis of a brain lesion, for example a metastatic cancer lesion, may be performed where the potential lesion [¹⁸F]-FACBC uptake is higher than both blood pool [¹⁸F]-FACBC uptake and parotid glands [¹⁸F]-FACBC uptake.

Diagnosis of the absence of a brain lesion, for example a metastatic cancer lesion may be performed where the potential lesion [¹⁸F]-FACBC uptake is not higher than either blood pool [¹⁸F]-FACBC uptake or parotid glands [¹⁸F]-FACBC uptake.

In some embodiments of the present disclosure, the potential lesion [¹⁸F]-FACBC uptake is visually compared to [¹⁸F]-FACBC activity in the blood pool and/or parotid glands to analyse and determine whether the potential lesion is a metastatic brain lesion in order to diagnose brain metastasis. In some embodiments of the present disclosure, [¹⁸F]-FACBC uptake higher than the blood pool is considered as suspicious for brain metastasis. In some embodiments of the present disclosure, [¹⁸F]-FACBC uptake higher than the parotid glands is considered as suspicious for brain metastasis. In some embodiments of the present disclosure, [¹⁸F]-FACBC uptake higher than both the blood pool and the parotid glands is considered as suspicious for brain metastasis. In some embodiments of the present disclosure, [¹⁸F]-FACBC uptake similar or less than blood pool is considered as non-suspicious for brain metastasis, e.g., indicative that the potential lesion is not a metastatic brain lesion. In some embodiments of the present disclosure, [¹⁸F]-FACBC uptake similar or less than the parotid glands is considered as non-suspicious for brain metastasis, e.g., indicative that the potential lesion is not a metastatic brain lesion.

In at least one aspect of the present disclosure there is provided a kit for imaging, diagnosing or monitoring metastatic cancer, comprising: a) [¹⁸F]-FACBC tracer; and b) administration instructions according to any of the aspects described above and/or elsewhere herein.

In at least one aspect of the present disclosure there is provided the acquisition of images using a PET/MRI or PET/CT scanner. The simultaneous or consecutive acquisition of images on a PET/MRI or PET/CT scanner may offer improved diagnostic accuracy over the generation of images on separate PET and MRI instruments or on separate PET and CT instruments. The conjoint use with MRI may improve the localisation of lesions, particularly where the lesions are small, for example in the case of metastatic lesions. The method of administration can be the methods described herein.

### BRIEF DESCRIPTION OF DRAWINGS

FIGS. 1a-1b show representative axial, sagittal and coronal [¹⁸F]-FACBC PET and fused PET/MRI images of the brain. FIG. 1a illustrates with arrows, the level of [¹⁸F]-FACBC activity within the blood pool, for comparison against [¹⁸F]-FACBC uptake in a potential lesion. FIG. 1b illustrates with arrows, the level of [¹⁸F]-FACBC activity within the parotid glands, for comparison against [¹⁸F]-FACBC uptake in a potential lesion.

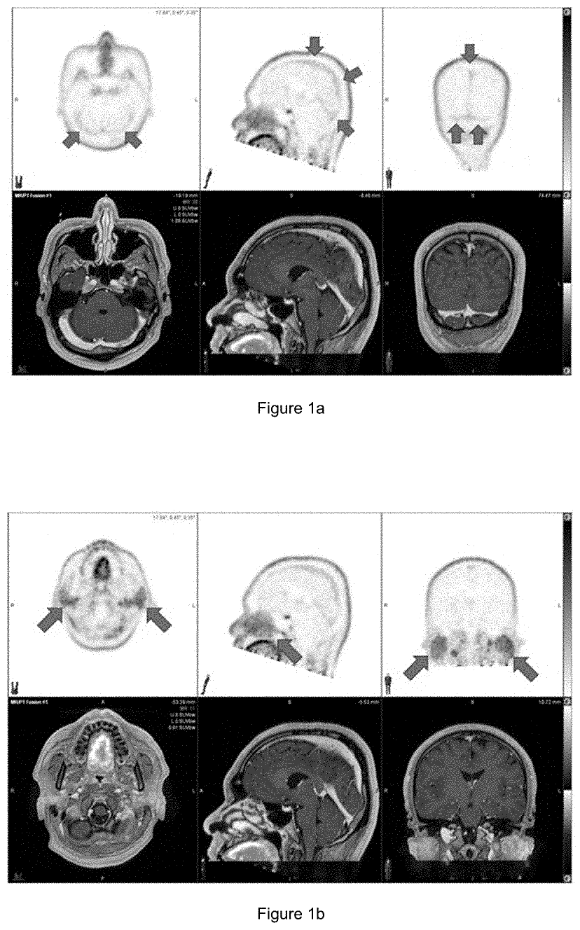

FIG. 2 shows representative coronal [¹⁸F]-FACBC PET and fused PET/MRI images of the brain. Upper arrow indicates [¹⁸F]-FACBC uptake in a potential lesion, higher than the level of [¹⁸F]-FACBC activity within the parotid glands (lower arrows).

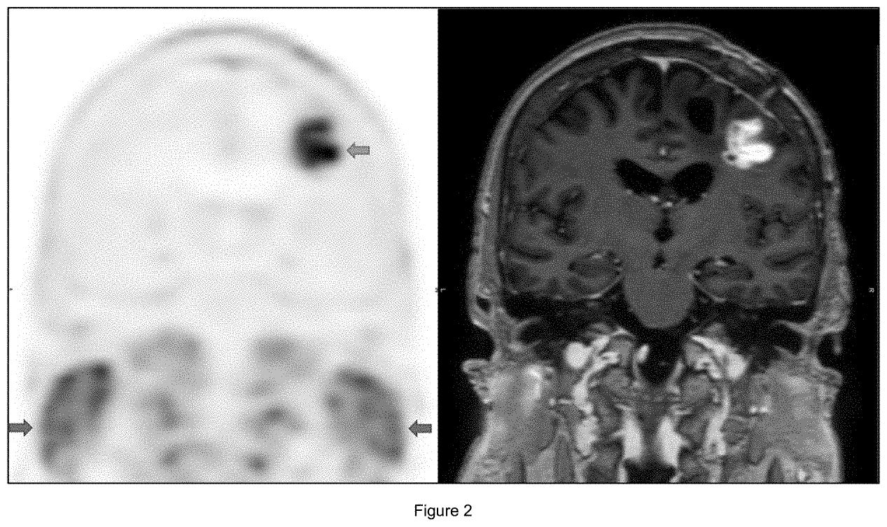

### DETAILED DESCRIPTION

The singular forms “a,” “an,” and “the” include plural reference unless the context dictates otherwise. The terms “approximately” and “about” refer to being nearly the same as a referenced number or value. As used herein, the terms “approximately” and “about” generally should be understood to encompass ±5% of a specified amount or value.

Fluciclovine (¹⁸F) injection, also known as [¹⁸F]-FACBC, FACBC, or anti-1-amino-3-¹⁸F-fluorocyclobutane-1-carboxylic acid, is a synthetic amino acid imaging agent which is taken up specifically by amino acid transporters and is used for positron emission tomography (PET). PET is uniquely suited to evaluate metabolic activity in human tissue for diagnostic imaging purposes. Use of ¹⁸F-fluciclovine in the imaging of brain metastases is of considerable clinical relevance. ¹⁸F-Fluciclovine has utility in the evaluation of primary and metastatic cancers in the brain due at least in part to its low normal brain background uptake and increased uptake by brain tumors.

The present inventors have established a protocol which allows for the imaging of brain lesions, e.g., providing greater reliability and reproducibility, allowing the data from PET scan images to be analysed and compared in order to accurately diagnose or monitor brain lesions. In at least one aspect of the present disclosure, visual comparison of the uptake of [¹⁸F]-FACBC uptake in a potential lesion against [¹⁸F]-FACBC uptake in the blood pool and/or the parotid glands allows a more accurate diagnosis of the lesion than using MRI and/or CT alone. An apparent greater uptake of a radiolabelled tracer is often not sufficient to determine accurately whether an area suspicious for cancer is indeed a brain lesion. Studies such as Bogsrud et al. (2019) have reported greater uptake by tumours compared to normal brain tissue, but the authors recognized limitations in accuracy based on tumour size and contrast. The present disclosure, including comparisons relative to blood pool and/or parotid glands, can provide for more accurate analyses of potential lesions to determine whether the potential lesions are indeed brain lesions, e.g., cancerous lesions. A potential lesion may include, for example, features on a CT or MRI scan image which are not conclusive on whether the features represent a brain lesion containing diseased tissue. Humans have two parotid glands located on either side of the mouth that are used for salivation. Prior reports using [¹⁸F]-FACBC in imaging have not investigated comparisons relative to parotid glands or relative to blood pool. As further described below, the methods according to the present disclosure using blood pool and/or parotid glands as a background reference can provide for more accurate diagnoses of the presence of a brain lesion, e.g., a cancerous brain lesion, such as a malignant or metastatic brain lesion. Visual comparison of the uptake of [¹⁸F]-FACBC uptake in a potential lesion against [¹⁸F]-FACBC uptake in the parotid glands may allow for a more accurate diagnosis of the lesion than using MRI and/or CT alone.

Semi-quantitative comparison of the uptake of [¹⁸F]-FACBC uptake in a potential lesion against a maximum standardized uptake value (SUVmax) threshold of 1 to 5.0 may allow for a more accurate diagnosis of the lesion than using MRI and/or CT alone. Semi-quantitative comparison of the uptake of [¹⁸F]-FACBC uptake in a potential lesion against a peak standardized uptake value (SUVpeak) threshold of 1 to 4.0 may allow for a more accurate diagnosis of the lesion than using MRI and/or CT alone. In some embodiments of the present disclosure, the SUVmax threshold is in a range of 1.0 to 5.0. In some embodiments of the present disclosure, for example, the SUVmax threshold may be in a range of 1.0 to 1.1, 1.1 to 1.2, 1.2 to 1.3, 1.3 to 1.4, 1.4 to 1.5, 1.5 to 1.6, 1.6 to 1.7, 1.7 to 1.8, 1.8 to 1.9, 1.9 to 2.0, 2.0 to 2.1, 2.1 to 2.2, 2.2 to 2.3, 2.3 to 2.4, 2.4 to 2.5, 2.5 to 2.6, 2.6 to 2.7, 2.7 to 2.8, 2.8 to 2.9, 2.9 to 3.0, 3.0 to 3.1, 3.1 to 3.2, 3.2 to 3.3, 3.3 to 3.4, 3.4 to 3.5, 3.5 to 3.6, 3.6 to 3.7, 3.7 to 3.8, 3.8 to 3.9, 3.9 to 4.0, 4.0 to 4.1, 4.1 to 4.2, 4.2 to 4.3, 4.3 to 4.4, 4.4 to 4.5, 4.5 to 4.6, 4.6 to 4.7, 4.7 to 4.8, 4.8 to 4.9, or 4.9 to 5.0. In some embodiments of the present disclosure, the SUVpeak threshold is in a range of 1.0 to 4.0. In some embodiments of the present disclosure, the SUVpeak threshold may be in a range of 1.0 to 1.1, 1.1 to 1.2, 1.2 to 1.3, 1.3 to 1.4, 1.4 to 1.5, 1.5 to 1.6, 1.6 to 1.7, 1.7 to 1.8, 1.8 to 1.9, 1.9 to 2.0, 2.0 to 2.1, 2.1 to 2.2, 2.2 to 2.3, 2.3 to 2.4, 2.4 to 2.5, 2.5 to 2.6, 2.6 to 2.7, 2.7 to 2.8, 2.8 to 2.9, 2.9 to 3.0, 3.0 to 3.1, 3.1 to 3.2, 3.2 to 3.3, 3.3 to 3.4, 3.4 to 3.5, 3.5 to 3.6, 3.6 to 3.7, 3.7 to 3.8, 3.8 to 3.9, or 3.9 to 4.0.

Disclosed is a method of detecting lesions in a subject, e.g., a patient, comprising administering [¹⁸F]-FACBC to the subject and imaging the subject on a PET/MRI or PET/CT scanner to obtain conjoint PET and MRI images or PET and CT images. The lesions may be metastatic lesions. The administration and/or PET imaging methods may be as described below and elsewhere herein.

In at least one aspect of the present disclosure, there is provided a method of using [¹⁸F]-FACBC, e.g., for diagnosing brain lesions, in a subject comprising the steps of:
(a) administering a detectable amount of [¹⁸F]-FACBC to the subject;
(b) acquiring one or more positron emission tomography (PET) scan images of a head region of the subject;
(c) comparing [¹⁸F]-FACBC uptake in a potential lesion against [¹⁸F]-FACBC uptake in the blood pool and/or the parotid glands; and
(d) determining whether the potential lesion is a brain lesion based on the analysis of step c);

wherein potential lesion [¹⁸F]-FACBC uptake which is higher than blood pool and/or parotid glands [¹⁸F]-FACBC uptake is indicative of the presence of one or more brain lesions.

In at least one aspect of the present disclosure, there is provided a method of using [¹⁸F]-FACBC, e.g., for diagnosing a brain lesion, in a subject, comprising the steps of:
a) administering a detectable amount of [¹⁸F]-FACBC to the subject;
b) acquiring one or more positron emission tomography (PET) scan images of a head region of the subject;
c) comparing [¹⁸F]-FACBC uptake in a potential lesion against [¹⁸F]-FACBC uptake in the blood pool and/or the parotid glands; and
d) determining whether the potential lesion is a brain lesion based on the analysis of step c);

wherein the subject is instructed to consume no food or calorie-containing drink during a given period of time prior to the administration of [¹⁸F]-FACBC; and

wherein the [¹⁸F]-FACBC uptake in the potential lesion which is higher than the [¹⁸F]-FACBC uptake in the blood pool and/or the parotid glands is indicative of the presence of the brain lesion.

In step a), [¹⁸F]-FACBC may be injected, e.g., as a bolus intravenous injection. The injection can be followed by a saline flush, e.g., a saline flush of about 10 mL or less.

After injection, lesion cells uptake [¹⁸F]-FACBC and the cells which have taken up the tracer can be subsequently visualised. Acquisition can start 8 to 12 minutes after the end of the injection, such as about 10 minutes after the end of the injection. Acquisition can start 8, 9, 10, 11 or 12 minutes after the end of the injection. [¹⁸F]-FACBC is generally taken up by lesion cells quickly compared to other PET radiotracers with different uptake mechanisms. For example, In FDG-PET imaging, acquisition usually starts at least 45 minutes after injection.

A “detectable amount” of [¹⁸F]-FACBC refers to a dosage of [¹⁸F]-FACBC which is taken up by lesion cells allowing those cells to be detected by PET imaging. In some examples, the dosage or detectable amount of [¹⁸F]-FACBC administered to the subject is 185±20% MBq. The dosage may be diluted up to 10 mL, e.g., via dilution with saline solution.

In at least one embodiment of the present disclosure, the subject, e.g., patient, is instructed to fast for a given period of time prior to the administration of [¹⁸F]-FACBC. The term “fast” means to consume no calories, e.g., to consume no food or calorie-containing drink. For example, only water, or other non-calorie-containing fluid, may be consumed during the given period of time prior to the administration of [¹⁸F]-FACBC. In some embodiments of the present disclosure, the given period of time is at least 6 hours. In some embodiments of the present disclosure, the given period of time is 4 to 6 hours. In some embodiments of the present disclosure, the given period of time is at least 4 hours. In some embodiments of the present disclosure, the given period of time is about 4 hours.

The PET imaging technique may utilise scanning devices that detect the 511 keV annihilation photons that are emitted after radioactive decay of fluorine-18. In addition, “micro-PET” scanners that have high spatial resolution can be used for imaging of small animals. In addition to PET scanners, ¹⁸F-radioactivity can also be monitored using one or more radiation detector probes.

In step b), the acquisition/scanning time can be for 8 to 12 minutes upon the start of acquisition, such as about 10 minutes upon the start of acquisition.

In some aspects there is provided a method of using [₁₈F]-FACBC, e.g., for imaging metastatic cancer, in a subject, comprising the steps of:
a) administering to the subject a first dose of [¹⁸F]-FACBC (e.g., a first dose of a detectable amount of [¹⁸F]-FACBC) and allowing time for the [¹⁸F]-FACBC to accumulate at one or more areas of interest within the subject;
b) obtaining a first PET scan image of the subject, e.g., obtaining a first PET scan image of a head region of the subject that includes parotid glands;
c) administering to the subject a second dose of [¹⁸F]-FACBC (e.g., a second dose of a detectable amount of [¹⁸F]-FACBC that is the same or different than the first dose) and allowing time for [¹⁸F]-FACBC to accumulate at the one or more areas of interest within the subject;
d) obtaining a second PET scan image of the subject, e.g., obtaining a second PET scan image of the head region of the subject;
e) comparing [¹⁸F]-FACBC uptake in a potential lesion against [¹⁸F]-FACBC uptake in the blood pool and/or the parotid glands for the first and second PET scan images, e.g., to determine whether the potential lesion is a brain lesion;

wherein the first and second scan images indicate the location and intensity of [¹⁸F]-FACBC and wherein localisation of [¹⁸F]-FACBC indicates the presence of lesion tissue in the subject.

In further aspects there is provided a method of using [¹⁸F]-FACBC, e.g., for diagnosing or monitoring metastatic cancer, in a subject, comprising the steps of:
a) administering to the subject a first dose of a detectable amount of [¹⁸F]-FACBC and allowing time for the [¹⁸F]-FACBC to accumulate at one or more areas of interest within the subject;
b) obtaining a first PET scan image of the subject, e.g., of a head region of the subject, wherein the first PET scan image indicates the location and intensity of [¹⁸F]-FACBC;
c) administering to the subject a second dose of a detectable amount of [¹⁸F]-FACBC and allowing time for [¹⁸F]-FACBC to accumulate at the one or more areas of interest within the subject;
d) obtaining a second PET scan image of the subject, e.g., of the head region of the subject, wherein the second PET scan image indicates the location and intensity of [¹⁸F]-FACBC;
e) comparing the first and second PET scan images to determine whether the location and/or intensity of [¹⁸F]-FACBC has increased, remained constant, or decreased, e.g., to determine whether the subject has metastatic cancer.

In at least one embodiment of the present disclosure, both PET and magnetic resonance imaging (MRI) or x-ray computed tomography (CT) scan images are acquired in steps b) and d), for example, by use of a combined PET-MRI or PET-CT system.

In at least one embodiment, [¹⁸F]-FACBC is administered according to the aspects of the present disclosure discussed above.

The time required for [¹⁸F]-FACBC to accumulate in lesion cells in steps a) and c) of the aspects described above may be about 10 minutes or less after [¹⁸F]-FACBC is administered. For example, the time taken for [¹⁸F]-FACBC to accumulate is 8 to 12 minutes, such as about 10 minutes. This therefore may allow image acquisition to start 8 to 12 minutes after administration, e.g., about 10 minutes after [¹⁸F]-FACBC administration.

During the PET scan, the subject, e.g., patient, may be in the supine position, with the head stabilised appropriately. The entire brain, including the cerebellum may be in the field of view. Thus, for example, the PET scan image may include a head region of the subject that includes the parotid glands.

Once data has been collected from the first PET scan, the images can be visualised and used to view the level, volume and/or location of lesion [¹⁸F]-FACBC uptake within the subject. Images are usually visually interpreted by a nuclear medicine physician or radiologist and standardised uptake values (SUVs) such as SUVmax and SUVpeak may be determined. The SUV is the ratio of tissue radioactivity concentration to the administered radiation dose divided by body weight. SUVmax is the highest intensity point, SUVpeak is the measurement within a defined volume. SUVmax is generally adversely affected by noise and variable between instruments, whereas SUVpeak can be a more robust alternative, but the associated volume of interest is not uniquely defined. SUVmax is higher than SUVpeak for any given PET scan.

The time between acquisition of the first and second PET scan images, i.e. between steps b) and d) of the aspects described above, can be as much as one year. In some instances, the time between the first and second PET scans is about 6 months, 5 months, 4 months, 3 months, 2 months, 1 month or even less than about 1 month. It will be appreciated that steps c) and d) of the aspects described above can be repeated as many times as necessary in order to obtain multiple scan images which can be used to map the development of a lesion over time.

Once image data has been collected from the second PET scan, the first and second PET scan images can be visualised together and used to view the change in extent and location of [¹⁸F]-FACBC uptake by a potential lesion within the subject, allowing for the diagnosis or monitoring of metastatic brain cancer. For example, if the level of lesion [¹⁸F]-FACBC uptake has changed then the subject may be diagnosed with metastatic cancer. In some embodiments, the second PET scan image can be compared to images of data collected from an earlier PET scan image taken before the first PET scan image, in addition to comparison with the first PET scan image. In addition, any subsequent PET scan images obtained after the second PET scan image can be compared with the first and/or second PET scan images.

By comparing the images from two or more differing time points, the differences in the potential lesion uptake of [¹⁸F]-FACBC can be analysed. Comparisons can involve either qualitative image comparison (e.g. contrast of potential lesion uptake to background) or quantitative indices derived from the imaging or external radiation detection data (e.g. SUVs). The development, progression or reduction of any lesions (or potential lesions) can therefore be monitored and diagnosed accordingly. Suitable treatment can then be determined, for example, targeted administration of localised treatment at the site of the lesion. It will be appreciated that the methods described herein can also be used to monitor response to various therapeutic regimens.

The PET scan images obtained in steps b) and d) of the methods described above may be combined with, preceded, and/or followed by anatomical imaging selected from computed tomography (CT) imaging, computerized axial tomography (CAT) imaging, or MRI. For combined imaging, the images can be acquired using a dedicated PET-CT, PET-MRI, or separate PET and CT/MRI scanning devices. If separate PET and CT/MRI imaging devices are used, image analysis techniques can be employed to spatially register the PET images with the anatomical images.

In at least one embodiment, the image acquisition steps b) and d) involve obtaining a combined PET scan image and MRI scan image. The PET-MRI images can be obtained using a dedicated PET-MRI scanning device. Such scanning devices are available from Siemens (Biograph mMR) and GE (SIGNA PET/MR). Since MRI does not use any ionizing radiation, its use is generally favoured in preference to CT. An advantage of PET-MRI acquisition is that the patient and medical staff generally only need to be present for a single scan resulting in a more time and cost efficient process.

In at least one embodiment, the image acquisition steps b) and d) involve obtaining a combined PET scan image and CT scan image. The PET-CT images can be obtained using a dedicated PET-CT scanning device. An advantage of PET-CT acquisition is that the patient and medical staff generally only need to be present for a single scan resulting in a more time and cost efficient process.

The methods described herein can be used for intra-organ mapping of lesion location, for example, the spatial distribution of brain lesion tissue within the brain can be determined for aiding in biopsy or treatment planning of the brain lesion.

The methods described herein may be performed prior to surgery to plan biopsy or surgical field, prior to radiotherapy to plan radiation field, after surgery, radiation or systemic treatment to assess response or plan subsequent treatment, to differentiate between treatment-related effects (including radiation necrosis/pseudoprogression) and recurrence of metastasis or as prognostic aid.

The methods described herein may be suitable for detecting metastasis formation derived from brain lesions. [¹⁸F]-FACBC can be used in the detection and localisation of a wide variety of metastatic cancers that may be present in the brain.

The methods described herein may be suitable for detecting recurrence of metastasis within brain lesions. [¹⁸F]-FACBC is particularly useful for imaging brain lesions.

In at least one embodiment, the methods herein are used to diagnose recurrent brain metastasis. For example, any of the methods above may be used for a subject previously diagnosed with brain metastasis.

The methods of the present disclosure have use in humans and some methods may have use in non-human animals (for example, dogs and cats). That is, the subject or patient may be a human or a non-human animal.

Aspects of the present disclosure provide a kit for imaging, diagnosing or monitoring metastatic cancer, comprising: a) [¹⁸F]-FACBC tracer; and b) administration instructions in accordance with any of the aspects of the present disclosure. For example, the administration instructions may include instructions for the patient to consume no food or calorie-containing drink during a given period of time prior to the administration of [¹⁸F]-FACBC. Additionally or alternatively, the administration instructions may include instructions for a medical professional to inject the [¹⁸F]-FACBC into the subject, e.g., as a bolus intravenous injection, optionally followed by a saline flush, such as a saline flush of about 10 mL or less. The instructions may include dosing information. Further, the administration instructions can include instructions for the medical professional to begin acquisition of a PET scan image 8 to 12 minutes after the end of the injection, such as about 10 minutes after the end of the injection.

Although the present disclosure has been described above with reference to exemplary embodiments, it is not intended to be limited to the specific form set forth herein. Rather, the invention is limited only by the accompanying claims and, other embodiments than those above are equally possible within the scope of these appended claims.

1. A method of using [¹⁸F]-FACBC, comprising the steps of:
(a) administering [¹⁸F]-FACBC to a subject, wherein the subject has followed instructions to consume no food or calorie-containing drink during a period of time prior to the administration of the [¹⁸F]-FACBC;
(b) acquiring one or more positron emission tomography (PET) scan images of a head region of the subject, the head region including parotid glands and a blood pool;
(c) analyzing the one or more PET scan images by comparing [¹⁸F]-FACBC uptake in a potential lesion of the head region of the subject against [¹⁸F]-FACBC uptake in the blood pool, or both the blood pool and the parotid glands; and
(d) determining whether the potential lesion is a brain lesion based on the analysis of step (c).

2. The method of claim 1, wherein the brain lesion is a cancer lesion.

3. The method of claim 2, wherein the cancer lesion is a metastatic cancer lesion.

4. The method of claim 3, wherein the [¹⁸F]-FACBC uptake in the potential lesion is higher than the [¹⁸F]-FACBC uptake in the parotid glands, and step (d) includes determining that the potential lesion is a brain lesion.

5. The method of claim 3, wherein the [¹⁸F]-FACBC uptake in the potential lesion is higher than the [¹⁸F]-FACBC uptake in the blood pool, and step (d) includes determining that the potential lesion is a brain lesion.

6. The method of claim 3, wherein the [¹⁸F]-FACBC uptake in the potential lesion is higher than both the [¹⁸F]-FACBC uptake in the blood pool and the [¹⁸F]-FACBC uptake in the parotid glands, and step (d) includes determining that the potential lesion is a brain lesion.

7. The method of claim 3, wherein the [¹⁸F]-FACBC uptake in the potential lesion is lower than or equal to the [¹⁸F]-FACBC uptake in the blood pool or the [¹⁸F]-FACBC uptake in the parotid glands, and step (d) includes determining that the potential lesion is not a brain lesion.

8. The method of claim 3, wherein the period of time is at least 6 hours.

9. The method of claim 3, wherein the period of time is at least 4 hours.

10. The method of claim 3, wherein acquiring the PET scan image starts 8 to 12 minutes after the administration of the [¹⁸F]-FACBC.

11. The method of claim 3, wherein acquiring the PET scan image starts 10 minutes after the administration of the [¹⁸F]-FACBC.

12. The method of claim 3, wherein the [¹⁸F]-FACBC is administered as an intravenous bolus injection.

13. The method of claim 3, wherein the amount of [¹⁸F]-FACBC administered to the subject is 185±20% MBq.

14. The method of claim 3, wherein the one or more PET scan images comprise a first PET scan image and at least one subsequent PET scan image, wherein analyzing the one or more PET scan images comprises:
comparing intensities of [¹⁸F]-FACBC uptake in the first PET scan image and the at least one subsequent PET scan image to determine whether the intensity of [¹⁸F]-FACBC uptake in the at least one subsequent PET scan image has increased, remained constant, or decreased relative to the intensity of [¹⁸F]-FACBC uptake of the first PET scan image.

15. The method of claim 3, wherein the one or more PET scan images comprises a PET scan image at a first location and at least one PET scan image at a subsequent location, and wherein analyzing the one or more PET scan images comprises:
comparing intensities of [¹⁸F]-FACBC uptake in the PET scan image at the first location and of [¹⁸F]-FACBC uptake in the at least one PET scan image at the subsequent location to determine whether the intensity of the [¹⁸F]-FACBC uptake in the at least one PET scan image at the subsequent location has increased, remained constant, or decreased relative to the intensity of the [¹⁸F]-FACBC uptake in the PET scan image at the first location.

16. The method of claim 1, wherein the potential lesion is suspicious for a recurrence of cancer in the subject.

17. A method of using [¹⁸F]-FACBC, comprising the steps of:
(a) administering [¹⁸F]-FACBC to a subject, wherein the subject has followed instructions to consume no food or calorie-containing drink during a period of time prior to the administration of the [¹⁸F]-FACBC;
(b) acquiring one or more positron emission tomography (PET) scan images of the head of the subject, the one or more PET scan images including parotid glands and a blood pool of the brain of the subject;
(c) analyzing the one or more PET scan images by comparing [¹⁸F]-FACBC uptake in a potential lesion of the brain of the subject against [¹⁸F]-FACBC uptake in the blood pool of the brain;
(d) determining whether the potential lesion is a brain lesion based on the analysis of step (c).

18. The method of claim 17, wherein step (c) further includes comparing [¹⁸F]-FACBC uptake in the potential lesion against [¹⁸F]-FACBC uptake in the parotid glands.

19. A method of using [¹⁸F]-FACBC, comprising the steps of:
(a) administering [¹⁸F]-FACBC to a subject previously diagnosed with brain metastasis, wherein the subject has followed instructions to consume no food or calorie-containing drink during a period of time prior to the administration of the [¹⁸F]-FACBC;
(b) acquiring one or more positron emission tomography (PET) scan images of a head region of the subject, the head region including parotid glands and a blood pool;
(c) analyzing the one or more PET scan images by comparing [¹⁸F]-FACBC uptake in a potential lesion of the head region of the subject against [¹⁸F]-FACBC uptake in the blood pool, or both the blood pool and the parotid glands; and
(d) determining whether the potential lesion is a brain lesion that includes diseased tissue based on the analysis of step (c).
